SEQUENCE LISTING (24 sequences)

SEQ ID NO: 1 - DNA, 19 bp, Artificial Sequence
  Synthetic construct  Polo-like kinase 1(Drosophila) sense primer
ctcaacacgcctcatcctc

SEQ ID NO: 2 - DNA, 20 bp, Artificial Sequence
  Synthetic construct  Polo-like kinase 1(Drosophila) antisense primer
gtgctcgctcatgtaattgc

SEQ ID NO: 3 - DNA, 22 bp, Artificial Sequence
  Synthetic construct  Cyclin B1 sense primer
tctggataatggtgaatggaca

SEQ ID NO: 4 - DNA, 22 bp, Artificial Sequence
  Synthetic construct  Cyclin B1 antisense primer
cgatgtggcatacttgttcttg

SEQ ID NO: 5 - DNA, 24 bp, Artificial Sequence
  Synthetic construct  Growth factor independent 1B sense primer
ttcctggtgaagagcaagaaggct

SEQ ID NO: 6 - DNA, 24 bp, Artificial Sequence
  Synthetic construct  Growth factor independent 1B antisense primer
tccaggcactggtttgggaataga

SEQ ID NO: 7 - DNA, 24 bp, Artificial Sequence
  Synthetic construct  Fatty acid desaturase 2 sense primer
tggatggaacagctaaggccaaga

SEQ ID NO: 8 - DNA, 24 bp, Artificial Sequence
  Synthetic construct  Fatty acid desaturase 2 antisense primer
ctgtggtttgcagccagatggttt

SEQ ID NO: 9 - DNA, 26 bp, Artificial Sequence
  Synthetic construct  Peroxisome proliferative activated receptor, gamma sense primer
cacaagaacagatccagtggttgcag

SEQ ID NO: 10 - DNA, 26 bp, Artificial Sequence
  Synthetic construct  Peroxisome \ proliferative activated receptor, gamma antisense primer
aataataaggtggagatgcaggctcc

SEQ ID NO: 11 - DNA, 24 bp, Artificial Sequence
  Synthetic construct  Stearoyl-CoA desaturase (delta-9-desaturase) sense primer
aacttgatacgtccgtgtgtccca

SEQ ID NO: 12 - DNA, 24 bp, Artificial Sequence
  Synthetic construct  Stearoyl-CoA desaturase (delta-9-desaturase) antisense primer
ctgtatgtttccgtggcaatgcgt

SEQ ID NO: 13 - DNA, 24 bp, Artificial Sequence
  Synthetic construct  sh2 domain containg 5 sense primer
agacctggtcattggtccagactt

SEQ ID NO: 14 - DNA, 24 bp, Artificial Sequence
  Synthetic construct  sh2 domain containg 5 antisense primer
aacatggccctgatagcttctcca

SEQ ID NO: 15 - DNA, 24 bp, Artificial Sequence
  Synthetic construct  Ankyrin repeat domain 1(cardiac muscle)sense primer
aagcgagaaacaacgagaggcaga

SEQ ID NO: 16 - DNA, 24 bp, Artificial Sequence
  Synthetic construct  Ankyrin repeat domain 1(cardiac muscle)antisense primer
agaaacgtaggcacatccacaggt

SEQ ID NO: 17 - DNA, 24 bp, Artificial Sequence
  Synthetic construct  Lymphocyte antigen 96 sense primer
agctctgaagggagagactgtgaa

SEQ ID NO: 18 - DNA, 24 bp, Artificial Sequence
  Synthetic construct  Lymphocyte antigen 96 antisense primer
ggtgtaggatgacaaactccaagc

SEQ ID NO: 19 - DNA, 24 bp, Artificial Sequence
  Synthetic construct  Growth differentiation factor 15 sense primer
aagaactcaggacggtgaatggct

SEQ ID NO: 20 - DNA, 24 bp, Artificial Sequence
  Synthetic construct  Growth differentiation factor 15 antisense primer
tttccgcaactctcggaatctgga

SEQ ID NO: 21 - DNA, 24 bp, Artificial Sequence
  Synthetic construct  Tumor necrosis factor receptor superfamily,member 14 sense primer
aggaatgtcagcaccagaccaagt

SEQ ID NO: 22 - DNA, 24 bp, Artificial Sequence
  Synthetic construct  Tumor necrosis factor receptor superfamily,member 14 antisense primer
ggccaactgtggagcaaacaatga

SEQ ID NO: 23 - DNA, 24 bp, Artificial Sequence
  Synthetic construct  DEP domain containing 1 sense primer
gccaccaagctgtggaatgaagtt

SEQ ID NO: 24 - DNA, 24 bp, Artificial Sequence
  Synthetic construct  DEP domain containing 1 antisense primer
atccactgcttctcctgctgtgaa

(19) United States
(12) Patent Application Publication
(10) Pub. No.: US 2009/0269747 A1
(43) Pub. Date: Oct. 29, 2009

(54) Marker Genes Based on Amiodarone Treatment for Screening of Drug Inducing Toxicity and Screening Method Therefor

(75) Inventors: Jae Chun Ryu, Seoul (KR); Youn Jung Kim, Seoul (KR); Mee Song, Seoul (KR)

Correspondence Address: GREENLEE WINNER AND SULLIVAN P C, 4875 PEARL EAST CIRCLE, SUITE 200, BOULDER, CO, 80301, US

(73) Assignee: Korea Institute of Science and Technology, Seoul (KR)

(21) Appl. No.: 12/121,724

(22) Filed: May 15, 2008

(30) Foreign Application Priority Data
Jul. 26, 2007 (KR) 10-2007-0074992

(51) Int. Cl. C12Q 1/68

(52) U.S. Cl. 435/6

(57) ABSTRACT

The present invention relates to a marker gene for screening of drug candidates inducing pulmonary toxicity and a screening method using the same, more precisely a marker gene up- or down regulated by amiodarone which is a drug inducing pulmonary toxicity and a method for screening drug candidates inducing pulmonary toxicity using the same. The marker gene of the present invention can be effectively used for monitoring and identifying drugs or chemical having high risk of inducing pulmonary toxicity and can be used as an effective tool for examining the mechanism of amiodarone which causes pulmonary toxicity and side effects.

### TECHNICAL FIELD

The present invention relates to a marker gene for screening a drug candidate inducing pulmonary toxicity and a screening method using the same, more precisely a marker gene up- or down regulated by amiodarone which is a drug inducing pulmonary toxicity and a method for screening a drug candidate inducing pulmonary toxicity using the same.

### BACKGROUND ART

Amiodarone has been used as an antidepressant and a therapeutic agent for arrhythmia and angina pectoris. It was reported that approximately 50% of the patients treated with amiodarone exhibited side effects and among those approximately 5-15% patients had pulmonary toxicity (Kaori Okayasu et al, Intern. Med, 45(22), 1303-7, 2006). 20-40% of the patients treated with amiodarone exhibited boredom, fatigue, tremor, walking difficulty and peripheral neuropathy. Approximately 25% of the patients treated with amiodarone showed gastrointestinal disorder, nausea, vomiting, constipation and loss of appetite. In addition, ophthalmic diseases such as optic neuropathy and optic neuritis can be induced and respiratory diseases such as pneumonia and pulmonary fibrosis can also be induced. Although it is not very often, hypothyroidism, insomnia, headache, arrhythmia and asthma can be induced. In as rare cases as approximately less than 1% of the patients treated with amiodarone, rash or other skin disorders changing skin color into blue gray, ecchymosis, alopecia, and hypotension can be induced.

It was also reported that phospholipidosis is the most representative lung injury related disease caused as side effect of amiodarone treatment. Phospholipidosis is related to lipid metabolism, which inhibits the activity of phospholipase A1 and phospholipase A2 in lysosomes of cells so that phospholipid is over-accumulated. Martin W J et al, J. Pharmacol. Exp. Ther, 251(1), 272-8, 1989) ASAH1N-acylsphingosine amidohydrolase (acid ceramidase) 1; MGC4171 hypothetical protein MGC4171; LSSlanosterol synthase (2,3-oxidosqualene-lanosterol cyclase); NR0B2nuclear receptor subfamily 0, group B, member 2; FABP1fatty acid binding protein 1, liver; HPNhepsin (transmembrane protease, serine 1); SERPINA3serine (or cysteine) proteinase inhibitor; clade A (alpha-1 antiproteinase, antitrypsin), member 3; C10orf10chromosome 10 open reading frame 10; FLJ10055hypothetical protein FLJ10055; FRCP1likely ortholog of mouse fibronectin type III repeat containing protein 1; SLC2A3solute carrier family 2 (facilitated glucose transporter) member 3; and TAGLNtransgelin. These genes have been identified as the phospholipidosis markers (H. Sawada et al. Toxicology in Vitro 20, 15061513, 2006).

Calcitonin gene-related peptide (CGRP) acts as an epithelial cell growth factor so that it plays a certain role in recovery of injured lung (Morimoto Y et al. Inhal Toxicol. March: 19(3): 283-9, 2007). In the meantime, c-Jun, a nuclear transcription factor induced by amiodarone and TGF-β1, a growth factor, allegedly cause pulmonary fibrosis (Chung W H et al. Am J Physiol Lung Cell Mol Physiol, November; 281(5): L1180-8, 2001).

Nucleotide sequencing project of genomes of various species including 6 species of mammals and 292 species of microorganisms has been completed and reported to NCBI (National Center for Biotechnology Information). Based on this huge data, genome-wide expression study has been undergoing to disclose functions of those genes. DNA microarray is also performed to analyze expressions of thousands of genes at a time (Schena, M., et al., Proc. Natl. Acad. Sci. USA 93: 10614-10619, 1996).

Microarray is prepared by integrating cDNA (complementary DNA) or 20-25 base pair long oligonucleotide sets on a glass substrate. cDNA microarray has been produced in laboratories and companies such as Agilent and Genomic Solutions, in which cDNAs are fixed by a mechanical method or ink jetting on a chip (Sellheyer, K. and Belbin, T. J., J. Am. Acad. Dermatol. 51: 681-692, 2004). Oligonucleotide microarray has been produced in Affymetrix by direct synthesis on a chip via photolithography and in Agilent by fixing synthetic oligonucleotides on a chip (Sellheyer, K. et. al., Am. Acad. Dermatol. 51: 681-692, 2004).

To analyze gene expression, RNAs are extracted from samples such as tissues, which are hybridized with oligonucleotide on DNA microarray. The obtained RNA is labeled with a fluorescent material or an isotope and converted into cDNA. In oligo-microarray, the control and the experimental groups are labeled with two different fluorescent materials (for example, Cy3 and Cy5), followed by hybridization simultaneously on the chip. Images are optically scanned to measure the strength of fluorescence and the results are analyzed. By comparing the strengths of the two different fluorescent materials, gene expression is determined (Somasundaram, K., et al., Genomics Proteomics I: 1-10, 2002).

The recent DNA microarray based high tech toxicogenomics enables the analysis and quantification of gene expression patterns in specific tissues or cell lines induced by novel a drug candidate and other chemicals. That is, by analyzing the expression frequency of a specific gene in a specific cell, it is possible to identify a gene causing side effects of such drugs. Then, it is further possible to understand the molecular mechanism related to such side effects and functions of drugs, leading to the screening and diagnosis of side effects and toxicity inducing materials.

The present inventors screened genes up-regulated or down-regulated by amiodarone by observing and analyzing gene expression profiles of amiodarone in BEAS-2B, a human bronchial epithelial cell line, using oligo-microarray on which 41,000 human genes are integrated, and further completed this invention by confirming a marker gene capable of detecting a drug candidate inducing pulmonary toxicity by investigating expressions of target genes by real-time RT-PCR and by establishing a method for screening a drug candidate inducing pulmonary toxicity.

### DISCLOSURE

### Technical Problem

It is an object of the present invention to provide a method for screening a drug candidate inducing pulmonary toxicity by using a marker gene whose expression is up-regulated or down-regulated by amiodarone, a drug inducing pulmonary toxicity.

### Technical Solution

To achieve the above object, The present invention provides a method for screening a drug candidate inducing pulmonary toxicity using a marker gene for a drug inducing pulmonary toxicity which changes its expression pattern in bronchial epithelial cells by amiodarone, and the drug inducing pulmonary toxicity.

Hereinafter, the present invention is described in detail.

The present invention provides a method for screening a drug candidate inducing pulmonary toxicity using a marker gene for a drug inducing pulmonary toxicity which changes its expression pattern by amiodarone, and the drug inducing pulmonary toxicity.

The drug candidate inducing pulmonary toxicity is preferably amiodarone, but not always limited thereto.

The marker gene herein is characteristically changed in its expression pattern by amiodarone in human bronchial epithelial cells.

The marker gene is composed of those genes involved in apoptosis, lipid metabolism, cell cycle, cell proliferation, and signal transduction or transcription.

The marker gene contains at least one gene selected from the group composed as follows: Genebank Accession Number: NM_{—}014391 [Ankyrin repeat domain 1 (cardiac muscle)], Genebank Accession Number: BC050651 (Brain expressed X-linked 2), Genebank Accession Number: AK128526 (Chromosome 9 open reading frame 58), Genebank Accession Number: BC000054 (7-dehydrocholesterol reductase), Genebank Accession Number: NM_{—}004265 (Fatty acid desaturase), Genebank Accession Number: NM_{—}004864 (Growth differentiation factor 15), Genebank Accession Number: (Genebank) AJ002231 (Glucosamine-6-phosphate deaminase 1), Genebank Accession Number: BC032783 (Glycoprotein (transmembrane) nmb), Genebank Accession Number: NM_{—}014707 (Histone deacetylase 9), Genebank Accession Number: T92260 (Transcribed locus), Genebank Accession Number: BE614991 [Transcribed locus, strongly similar to XP_{—}514491.1 PREDICTED: similar to hypothetical protein (Pan troglodytes)], Genebank Accession Number: BE614991 [Transcribed locus, strongly similar to XP_{—}514491.1 PREDICTED: similar to hypothetical protein (Pan troglodytes)], Genebank Accession Number: AF026246, Genebank Accession Number: NM_{—}006850 (Interleukin 24), Genebank Accession Number: NM_{—}198336 (Insulin induced gene 1), Genebank Accession Number: XM_{—}044178 (KIAA1211 protein), Genebank Accession Number: NM_{—}014079 (Kruppel-like factor 15), Genebank Accession Number: NM_{—}005559 (Laminin, alpha 1), Genebank Accession Number: AK090454 (Family with sequence similarity 59, member B), Genebank Accession Number: AK094730 (Hypothetical protein LOC283454), Genebank Accession Number: AK124869 (Hypothetical protein LOC149194), Genebank Accession Number: BX537968 (Hypothetical LOC51149), Genebank Accession Number: BM918324 (Lymphocyte antigen 96), Genebank Accession Number: CR617492 (Family with sequence similarity 89, member A), Genebank Accession Number: NM_{—}007289 [Membrane metallo-endopeptidase (neutral endopeptidase, enkephalinase, CALLA, CD10)], Genebank Accession Number: BC013875 [Matrix metallopeptidase 1 (interstitial collagenase)], Genebank Accession Number: BG284742 [P8 protein (candidate of metastasis 1)], Genebank Accession Number: AK124635 (Proprotein convertase subtilisin/kexin type 9), Genebank Accession Number: NM_{—}138711 (Peroxisome proliferative activated receptor, gamma), Genebank Accession Number: BX648997 (Peroxisome proliferative activated receptor, gamma, coactivator 1, beta), Genebank Accession Number: NM_{—}003621 [PTPRF interacting protein, binding protein 2 (liprin beta 2)], Genebank Accession Number: XM_{—}350880 [Protein phosphatase 1H (PP2C domain containing)], Genebank Accession Number: BC033883 (Retinol binding protein 7, cellular), Genebank Accession Number: AL137502 (Ras-related GTP binding D), Genebank Accession Number: NM_{—}005063 [Stearoyl-CoA desaturase (delta-9-desaturase)], Genebank Accession Number: NM_{—}003627 (Solute carrier family 43, member 1), Genebank Accession Number: NM_{—}012449 (Six transmembrane epithelial antigen of the prostate 1), Genebank Accession Number: AL834346 [Syntaxin binding protein 6 (amisyn)], Genebank Accession Number: AL832142 [Transmembrane 7 superfamily member 1 (upregulated in kidney)], Genebank Accession Number: NM_{—}003820 [Tumor necrosis factor receptor superfamily, member 14 (herpesvirus entry mediator)], Genebank Accession Number: NM_{—}033035 (Thymic stromal lymphopoietin), Genebank Accession Number: AW665665 [Transcribed locus, strongly similar to XP_{—}509406.1 PREDICTED: similar to hypothetical protein FLJ14627 (Pan troglodytes)], Genebank Accession Number: NM_{—}003377 (Vascular endothelial growth factor B), EnsEMBL Accession Number: ENST00000313481, Genebank Accession Number: NM_{—}170589 (Cancer susceptibility candidate 5), Genebank Accession Number: BC053619 (Arrestin domain containing 3), Genebank Accession Number: NM_{—}000046 (Arylsulfatase B), Genebank Accession Number: AY367065 [Asp (abnormal spindle)-like, microcephaly associated (Drosophila)], Genebank Accession Number: NM_{—}052876 [BTB (POZ) domain containing 14B], Genebank Accession Number: NM_{—}031966 (Cyclin B1), Genebank Accession Number: NM_{—}001813 (Centromere protein E, 312 kDa), Genebank Accession Number: BC036307 (Calponin 1, basic, smooth muscle), Genebank Accession Number: M28016 (Human mitochondrial cytochrome b gene, partial cds.), Genebank Accession Number: BC065304 (DEP domain containing 1), Genebank Accession Number: AB209653 [Dehydrogenase/reductase (SDR family) member 2], Genebank Accession Number: NM_{—}001964 (Early growth response 1), Genebank Accession Number: AK122613 (ATPase type 13A5), Genebank Accession Number: CR600908 [full-length cDNA clone CS0DL005YJ22 of B cells (Ramos cell line) Cot 25-normalized of Homo sapiens (human).], Genebank Accession Number: BC043371 [Growth factor independent 1B (potential regulator of CDKN1A, translocated in CML)], Genebank Accession Number: NM_{—}016548 (Golgi phosphoprotein 2), Genebank Accession Number: NM_{—}005319 (Histone 1, H1c), Genebank Accession Number: NM_{—}003512 (Histone 1, H2ac), Genebank Accession Number: BC082232 (Histone 1, H2bg), Genebank Accession Number: BC101655 (Histone 1, H2bi), Genebank Accession Number: NM_{—}080593 (Histone 1, H2bk), Genebank Accession Number: BM752802 (Histone 1, H2bm), Genebank Accession Number: BX647290 (Histone 1, H2bo), Genebank Accession Number: BC069193 (Histone 2, H2be), Genebank Accession Number: AF032862 [Hyaluronan-mediated motility receptor (RHAMM)], Genebank Accession Number: BE620675 [Transcribed locus, moderately similar to NP_{—}536855.1 cytochrome b (Homo sapiens)], Genebank Accession Number: AY358369 [PRO333; Homo sapiens clone DNA41374 SIGLEC5 (UNQ294) mRNA, partial cds.], Genebank Accession Number: BE300829 [Transcribed locus, moderately similar to NP_{—}005021.2 polo-like kinase; polo (Drosophila)-like kinase; polo-like kinase (Drosophila) (Homo sapiens)], Genebank Accession Number: AY791349 (Kinesin family member 18A), Genebank Accession Number: AK025790 (Kinesin family member 20A), Genebank Accession Number: BC029844 (Hypothetical protein LOC256021), Genebank Accession Number: AF001540 [metastasis associated lung adenocarcinoma transcript 1 (non-coding RNA)], Genebank Accession Number: NM_{—}001620 [AHNAK nucleoprotein (desmoyokin)], Genebank Accession Number: NM_{—}00593 [Myeloid/lymphoid or mixed-lineage leukemia (trithorax homolog, Drosophila)], Genebank Accession Number: NM_{—}012333 (C-myc binding protein), Genebank Accession Number: AJ002535 (Obscurin, cytoskeletal calmodulin and titin-interacting RhoGEF), Genebank Accession Number: AB209179 [Polo-like kinase 1 (Drosophila)], Genebank Accession Number: NM_{—}006904 (Protein kinase, DNA-activated, catalytic polypeptide), Genebank Accession Number: BC050630 [RAB3 GTPase activating protein subunit 2 (non-catalytic)], Genebank Accession Number: AB095943 (SNF2 histone linker PHD RING helicase), Genebank Accession Number: AB016092 (Serine/arginine repetitive matrix 2), Genebank Accession Number: NM_{—}006472 (Thioredoxin interacting protein), Genebank Accession Number: DQ097177 (HECT, UBA and WWE domain containing 1), Genebank Accession Number: NM_{—}016267 [Vestigial like 1 (Drosophila)], TIGR (The Institute for Genomic Research) Accession Number: THC2428713.

Marker genes up-regulated by the treatment of a drug inducing pulmonary toxicity are as follows:

Genebank Accession Number: NM_{—}014391 [Ankyrin repeat domain 1 (cardiac muscle)], Genebank Accession Number: BC050651 (Brain expressed X-linked 2), Genebank Accession Number: AK128526 (Chromosome 9 open reading frame 58), Genebank Accession Number: BC000054 (7-dehydrocholesterol reductase), Genebank Accession Number: NM_{—}004265 (Fatty acid desaturase), Genebank Accession Number: NM_{—}004864 (Growth differentiation factor 15), Genebank Accession Number: AJ002231 (Glucosamine-6-phosphate deaminase 1), Genebank Accession Number: BC032783 (Glycoprotein (transmembrane) nmb), Genebank Accession Number: NM_{—}014707 (Histone deacetylase 9), Genebank Accession Number: T92260 (Transcribed locus), Genebank Accession Number: BE614991 [Transcribed locus, strongly similar to XP_{—}514491.1 PREDICTED: similar to hypothetical protein (Pan troglodytes)], Genebank Accession Number: BE614991 [Transcribed locus, strongly similar to XP_{—}514491.1 PREDICTED: similar to hypothetical protein (Pan troglodytes)], Genebank Accession Number: AF026246, Genebank Accession Number: NM_{—}006850 (Interleukin 24), Genebank Accession Number: NM_{—}198336 (Insulin induced gene 1), Genebank Accession Number: XM_{—}044178 (KIAA1211 protein), Genebank Accession Number: NM_{—}014079 (Kruppel-like factor 15), Genebank Accession Number: NM_{—}005559 (Laminin, alpha 1), Genebank Accession Number: AK090454 (Family with sequence similarity 59, member B), Genebank Accession Number: AK094730 (Hypothetical protein LOC283454), Genebank Accession Number: AK124869 (Hypothetical protein LOC149194), Genebank Accession Number: BX537968 (Hypothetical LOC51149), Genebank Accession Number: BM918324 (Lymphocyte antigen 96), Genebank Accession Number: CR617492 (Family with sequence similarity 89, member A), Genebank Accession Number: NM_{—}007289 [Membrane metallo-endopeptidase (neutral endopeptidase, enkephalinase, CALLA, CD10)], Genebank Accession Number: BC013875 [Matrix metallopeptidase 1 (interstitial collagenase)], Genebank Accession Number: BG284742 [P8 protein (candidate of metastasis 1)], Genebank Accession Number: AK124635 (Proprotein convertase subtilisin/kexin type 9), Genebank Accession Number: NM_{—}138711 (Peroxisome proliferative activated receptor, gamma), Genebank Accession Number: BX648997 (Peroxisome proliferative activated receptor, gamma, coactivator 1, beta), Genebank Accession Number: NM_{—}003621 [PTPRF interacting protein, binding protein 2 (liprin beta 2)], Genebank Accession Number: XM_{—}350880 [Protein phosphatase 1H (PP2C domain containing)], Genebank Accession Number: BC033883 (Retinol binding protein 7, cellular), Genebank Accession Number: AL137502 (Ras-related GTP binding D), Genebank Accession Number: NM_{—}005063 [Stearoyl-CoA desaturase (delta-9-desaturase)], Genebank Accession Number: NM_{—}003627 (Solute carrier family 43, member 1), Genebank Accession Number: NM_{—}012449 (Six transmembrane epithelial antigen of the prostate 1), Genebank Accession Number: AL834346 [Syntaxin binding protein 6 (amisyn)], Genebank Accession Number: AL832142 [Transmembrane 7 superfamily member 1 (upregulated in kidney)], Genebank Accession Number: NM_{—}003820 [Tumor necrosis factor receptor superfamily, member 14 (herpesvirus entry mediator)], Genebank Accession Number: NM_{—}033035 (Thymic stromal lymphopoietin), Genebank Accession Number: AW665665 [Transcribed locus, strongly similar to XP_{—}509406.1 PREDICTED: similar to hypothetical protein FLJ14627 (Pan troglodytes)], Genebank Accession Number: NM_{—}003377 (Vascular endothelial growth factor B), EnsEMBL Accession Number: ENST00000313481.

Marker genes down-regulated by the treatment of a drug inducing pulmonary toxicity are as follows:

Genebank Accession Number: NM_{—}170589 (Cancer susceptibility candidate 5), Genebank Accession Number: BC053619 (Arrestin domain containing 3), Genebank Accession Number: NM_{—}000046 (Arylsulfatase B), Genebank Accession Number: AY367065 [Asp (abnormal spindle)-like, microcephaly associated (Drosophila)], Genebank Accession Number: NM_{—}052876 [BTB (POZ) domain containing 14B], Genebank Accession Number: NM_{—}031966 (Cyclin B1), Genebank Accession Number: NM_{—}001813 (Centromere protein E, 312 kDa), Genebank Accession Number: BC036307 (Calponin 1, basic, smooth muscle), Genebank Accession Number: M28016 (Human mitochondrial cytochrome b gene, partial cds.), Genebank Accession Number: BC065304 (DEP domain containing 1), Genebank Accession Number: AB209653 [Dehydrogenase/reductase (SDR family) member 2], Genebank Accession Number: NM_{—}001964 (Early growth response 1), Genebank Accession Number: AK122613 (ATPase type 13A5), Genebank Accession Number: CR600908 [full-length cDNA clone CS0DL005YJ22 of B cells (Ramos cell line) Cot 25-normalized of Homo sapiens(human).], Genebank Accession Number: BC043371 [Growth factor independent 1B (potential regulator of CDKN1A, translocated in CML)], Genebank Accession Number: NM_{—}016548 (Golgi phosphoprotein 2), Genebank Accession Number: NM_{—}005319 (Histone 1, H1c), Genebank Accession Number: NM_{—}003512 (Histone 1, H2ac), Genebank Accession Number: BC082232 (Histone 1, H2bg), Genebank Accession Number: BC101655 (Histone 1, H2bi), Genebank Accession Number: NM_{—}080593 (Histone 1, H2bk), Genebank Accession Number: BM752802 (Histone 1, H2bm), Genebank Accession Number: BX647290 (Histone 1, H2bo), Genebank Accession Number: BC069193 (Histone 2, H2be), Genebank Accession Number: AF032862 [Hyaluronan-mediated motility receptor (RHAMM)], Genebank Accession Number: BE620675 [Transcribed locus, moderately similar to NP_{—}536855.1 cytochrome b (Homo sapiens)], Genebank Accession Number: AY358369 [PRO333; Homo sapiens clone DNA41374 SIGLEC5 (UNQ294) mRNA, partial cds.], Genebank Accession Number: BE300829 [Transcribed locus, moderately similar to NP_{—}005021.2 polo-like kinase; polo (Drosophila)-like kinase; polo-like kinase (Drosophila)(Homo sapiens)], Genebank Accession Number: AY791349 (Kinesin family member 18A), Genebank Accession Number: AK025790 (Kinesin family member 20A), Genebank Accession Number: BC029844 (Hypothetical protein LOC256021), Genebank Accession Number: AF001540 [metastasis associated lung adenocarcinoma transcript 1 (non-coding RNA)], Genebank Accession Number: NM_{—}001620 [AHNAK nucleoprotein (desmoyokin)], Genebank Accession Number: NM_{—}00593 [Myeloid/lymphoid or mixed-lineage leukemia (trithorax homolog, Drosophila)], Genebank Accession Number: NM_{—}012333 (C-myc binding protein), Genebank Accession Number: AJ002535 (Obscurin, cytoskeletal calmodulin and titin-interacting RhoGEF), Genebank Accession Number: AB209179 [Polo-like kinase 1 (Drosophila)], Genebank Accession Number: NM_{—}006904 (Protein kinase, DNA-activated, catalytic polypeptide), Genebank Accession Number: BC050630 [RAB3 GTPase activating protein subunit 2 (non-catalytic)], Genebank Accession Number: AB095943 (SNF2 histone linker PHD RING helicase), Genebank Accession Number: AB016092 (Serine/arginine repetitive matrix 2), Genebank Accession Number: NM_{—}006472 (Thioredoxin interacting protein), Genebank Accession Number: DQ097177 (HECT, UBA and WWE domain containing 1), Genebank Accession Number: NM_{—}016267 [Vestigial like 1 (Drosophila)], TIGR Accession Number: THC2428713.

To screen a marker gene for a drug inducing pulmonary toxicity, the present inventors treated amiodarone inducing pulmonary toxicity to a human bronchial epithelial cell line (BEAS-2B), followed by investigation of cytotoxicity. As a result, it was confirmed that amiodarone had toxicity to the human bronchial epithelial cell line (see FIG. 1), and based on this result, the amiodarone concentration for the treatment was determined. The human bronchial epithelial cell line was treated with amiodarone by the determined concentration and mRNA was extracted therefrom. During cDNA synthesis, it was labeled with Cy5. The control group not treated with the drug was labeled with Cy3. The fluorescence-labeled cDNA was hybridized with the 44k human whole genome oligo-microarray chip (Agilent, USA), followed by scanning of fluorescent images to analyze the gene expression patterns (see FIG. 2). When the margin of the ratio of Cy5 to Cy3 was more than 2.0 fold, the gene expression was considered to be increased so that the gene was classified into the up-regulated gene group. When the margin of the ratio of Cy5 to Cy3 was less than 0.5 fold, the gene expression was considered to be decreased so that the gene was classified into the down-regulated gene group. As a result, the gene identified to be up-regulated was 0.1% (44 genes out of 44,290 genes) and the gene identified to be down-regulated was 0.10% (45 genes out of 44,290 genes). At this time, the genes exhibiting 2.0 fold higher or less expression by amiodarone were classified according to the functions so that the genes having the functions involved in pulmonary toxicity, that is genes involved in apoptosis, lipid metabolism, cell cycle, cell proliferation, and signal transduction or transcription were selected (see Table 2 and Table 3). There have been no reports that those selected genes are involved in cytotoxicity in human bronchial epithelial cells according to the amiodarone treatment.

In examples of the present invention, three cell cycle related genes, 3 lipid metabolism-related genes and 7 signal transduction-related genes were separated, followed by real-time reverse transcript polymerase chain reaction (RT-PCR) to investigate expression patterns thereof. As a result, 8 up-regulated genes and 4 down-regulated genes were detected, consistent with the result of the experiment using the oligo-microarray chip (see Table 5).

The present invention also provides a method for screening a drug candidate inducing pulmonary toxicity comprising the following steps:

1) treating human bronchial epithelial cells with a sample compound or composition as an experimental group;

2) comparing expression level of a group of genes whose expression is altered by treatment of amiodarone between the experimental group of step 1) and a control group which are human bronchial epithelial cells untreated with the sample compound or composition; and

3) selecting the compound or composition which alters the expression level of the group of the genes significantly comparing to the control group.

In the above method, the human bronchial epithelial cells of step 1) are preferably BEAS-2B cells, but not always limited thereto and any human bronchus or lung originated cells can be used.

In the above method, the group of genes of step 2) is preferably the marker gene, but not always limited thereto and any gene altered by treatment of amiodarone can be used.

In the above method, the expression level of step 2) is preferably measured as mRNA level or protein level, but not always limited thereto.

The mRNA level is preferably measured by oligonucleotide or polynucleotide microarray, or RT-PCR, but not always limited thereto.

The protein level is preferably measured by protein microarray or ELISA, or RT-PCR, but not always limited thereto.

The present invention also provides a method for screening a drug candidate inducing pulmonary toxicity comprising the following steps:

1) treating human bronchial epithelial cells with a sample compound or composition as an experimental group;

2) extracting RNAs from the experimental group of step 1) and from the control group which are human bronchial epithelial cells untreated with the sample compound or composition;

3) converting RNAs extracted from the experimental group and the control group of step 2) into cDNA and labeling them with different fluorescent materials;

4) hybridizing cDNAs labeled with different fluorescent materials of step 3) with a DNA microarray containing a group of genes whose expression is altered by treatment of amiodarone;

5) analyzing the reacted DNA microarray of step 4);

6) comparing the expression level of a group of genes whose expression is altered by treatment of amiodarone between the experimental group and a control group based on the data analyzed in step 5); and

7) selecting the compound or composition which alters the expression level of the group of the genes significantly comparing to the control group.

In the above method, the human bronchial epithelial cells of step 1) are preferably BEAS-2B cells, but not always limited thereto and any human bronchus or lung originated cells can be used.

In the above method, the fluorescent material of step 3) is preferably selected from the group consisting of Cy3, Cy5, poly L-lysine-fluorescein isothiocyanate (FITC), rhodamine-B-isothiocyanate (RITC) and rhodamine, but not always limited thereto and any fluorescent material known to those in the art can be used.

In the above method, the group of genes of step 4) is preferably the marker gene, but not always limited thereto and any gene altered by treatment of amiodarone can be used.

In the above method, the DNA microarray of step 4) is preferably oligonucleotide or polynucleotide microarray, but not always limited thereto.

The DNA microarray is the whole human genome oligo microarray (Agilent, USA), but not always limited thereto, and any microarray that is loaded with those genes supposed to be up-regulated or down-regulated (see Table 2 and Table 3), among human genomes, can be used. In a preferred embodiment of the present invention, the DNA microarray chip prepared by the present inventors was used. And for the analysis of step 5), GenePix 4.1 software (Axon Instruments, USA) is preferably used, but not always limited thereto, and any software for analysis known to those in the art can be used.

In addition, the present invention provides a method for screening a drug candidate inducing pulmonary toxicity comprising the following steps:

1) treating human bronchial epithelial cells with a sample compound or composition as an experimental group;

2) extracting RNAs from the experimental group of step 1) and from the control group which are human bronchial epithelial cells untreated with the sample compound or composition;

3) performing real time RT-PCR with the RNAs of step 2) using primers corresponding to a group of genes whose expression is altered by treatment of amiodarone and capable of amplifying the group of genes;

4) comparing expression level of the group of genes between the experimental group and a control group; and

5) selecting the compound or composition which alters the expression level of the group of the genes significantly comparing to the control group.

In the above method, the human bronchial epithelial cells of step 1) are preferably BEAS-2B cells, but not always limited thereto and any human bronchus or lung originated cells can be used.

In the above method, the group of genes of step 3) is preferably the marker gene, but not always limited thereto and any gene altered by treatment of amiodarone can be used.

In the above method, the primers of step 3) are complementary to the marker genes screened in this invention and are capable of amplifying the marker genes and are designed to produce PCR product of 100-300 bp (base pairs). In this invention, forward primers and reverse primers represented by SEQ. ID. NO: 1-NO: 24 are proposed, but not always limited thereto.

### ADVANTAGEOUS EFFECT

The marker gene for screening of a drug candidate inducing pulmonary toxicity and the screening method of a drug candidate inducing pulmonary toxicity using the same are very effective and useful for monitoring drugs or chemicals having high risks or judging the risks thereof by using reacting genes selected by the DNA microarray chip as marker genes and are effective tools for disclosing functional mechanism of amiodarone causing pulmonary toxicity and side effects.

### DESCRIPTION OF DRAWINGS

The application of the preferred embodiments of the present invention is best understood with reference to the accompanying drawings, wherein:

FIG. 1 is a graph illustrating the cytotoxicity of amiodarone in a human bronchial epithelial cell line.

FIG. 2 is a diagram illustrating the gene expression pattern in the human bronchial epithelial cell line treated with amiodarone, investigated by using the microarray chip.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Cell Culture and Chemical Treatment

### <1-1> Cell Culture

BEAS-2B (CRL-9609, ATCC, USA), a human bronchial epithelial cell line, was cultured in a T 75 flask containing DMEM (Gibco-BRL, USA) supplemented with 10% FBS at the concentration of 5×10⁵ cells/ml. The present inventors selected amiodarone as a representative drug inducing pulmonary toxicity as side effects based on the previous studies and reports and dissolved the drug in DMSO. The concentration of the vehicle was less than 0.1% in every experiment.

### <1-2> Cytotoxicity Assay (MTT Assay) and Chemical Treatment

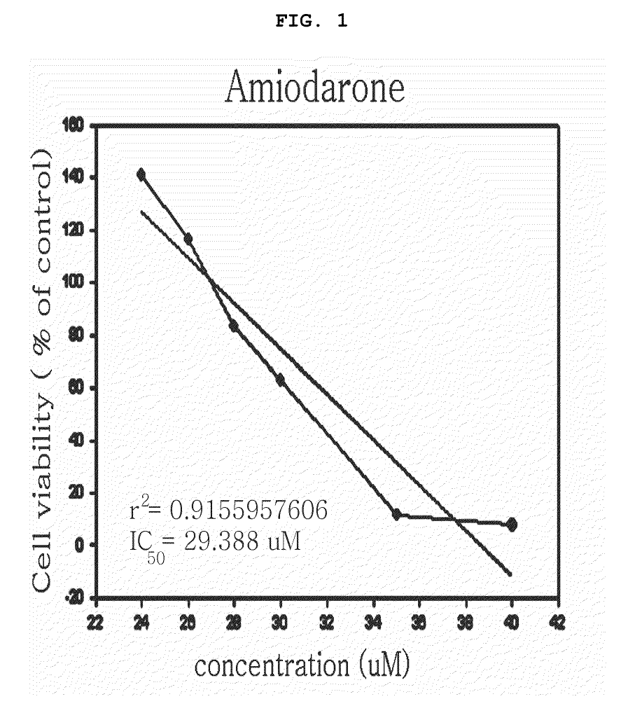

MTT assay with the BEAS-2B cell line was performed according to the method of Mossman et al (J. Immunol. Methods, 65, 55-63, 1983). The cells were inoculated in a 24-well plate containing DMEM (Gibco-BRL, USA) at the concentration of 3×10⁴ cells/well, to which amiodarone dissolved in DMSO was treated. 48 hours later, MTT (3-[4,5-dimethylthiazol-2-yl]-2,5-diphenyltetrazolium bromide) 5 mg/ml was added at the concentration of 5 mg/Ml, followed by culture at 37□ for 3 hours. Then, medium was eliminated and generated formazan crystal was dissolved in 500 μl of DMSO. The solution was aliquoted in a 96 well-plate and OD₅₄₀ was measured. Cytotoxicity in the BEAS-2B cell line caused by amiodarone was investigated. As a result, IC₂₀ was 29.388 uM (see FIG. 1) and thus the concentration of amiodarone for the further experiment was determined as the above.

### Example 2

### Microarray Experiment

<2-1> Separation of a Target RNA and Labeling with a Fluorescent Material

BEAS-2B cells were distributed on a 100 mm dish at the concentration of 1.24×10⁶ cell/Ml, to which amiodarone was treated for 48 hours at the concentration that was determined in Example <1-2>. Total RNA was extracted from the cells using trizol reagent (Invitrogen life technologies, USA) according to the manufacturer's instruction and the extracted RNA was purified by using RNeasy mini kit (Qiagen, USA). The genomic DNA was eliminated during the purification of RNA using RNase-free DNase set (Qiagen, USA). The total RNA was quantified by spectrophotometer and purity was measured by Agilent 2100 Bioanalyzer (Agilent Technologies, USA).

<2-2> Preparation of Labeled cDNA

For oligo-microarray analysis, cDNA was synthesized using the total RNA extracted from the experimental group treated with amiodarone obtained in Example <2-1>. 30 μg of the total RNA and 2 μg (1 μg/μl) of oligo (dT) primer were mixed, followed by reaction for 10 minutes at 65□. Then, the reaction mixture was put in ice, followed by annealing. Reagents were mixed as shown in Table 1 for reverse transcription of the RNA.

The total RNA extracted from the control BEAS-2B cell line was labeled with Cy3-dUTP (green), while the RNA extracted from the experimental BEAS-2B cell line treated with amiodarone was labeled with Cy5-dUTP (red). At this time, the two samples were mixed and purified by Microcon YM-30 column (Millipore, USA).

TABLE 1

| Composition | volume (μl) |
|---|---|
| 5X first strand | 6 |
| buffer | |
| dNTPs | 0.6 |
| 0.1 M DDT | 3 |
| SuperScript II | 3 |
| enzyme | |
| Cy-3 or Cy-5 dUTP | 2 |

### <2-3> Hybridization

Hybridization and washing were performed according to the instruction of GeneCneck Co., Ltd. Hybridization was performed in a 62□ oven for 12 hours. At this time, 44 k whole human genome oligo microarray (Agilent, USA) was used as a DNA microarray chip. After washing (with 2×SSC/0.1% SDS for 2 minutes, with 1×SSC for 3 minutes, with 0.2×SSC for 2 minutes), the slide was centrifuged at 800 rpm for 3 minutes and dried.

### <2-4> Fluorescence Image Obtaining

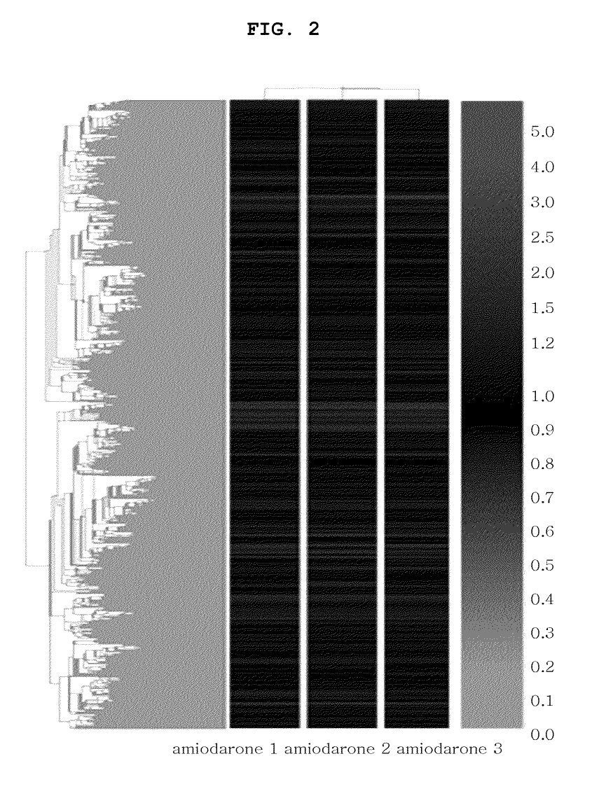

Scanning of the hybridized images on the slide was performed by using Genepix 4000B (Axon Instruments, USA). Fluorescence images of the chip washed to eliminate non-binding genes were obtained by laser fluorescence scanner. At this time, green fluorescent images indicated the activity of the gene expressed in the control, whereas red fluorescent images indicated the activity of the gene expressed specifically in the experimental group. In the meantime, yellow fluorescent images (complementary color of red and green) indicated that there was not much difference in the expression between the two groups. The scanned images were analyzed by GenePix 4.1 software (Axon Instruments, USA) to calculate the gene expression rate. Based on the obtained data, marker genes for amiodarone were selected (see FIG. 2).

As a result, the gene identified to be up-regulated was 0.1% (44 genes out of 44,290 genes) and the gene identified to be down-regulated was 0.10% (45 genes out of 44,290 genes) among approximately 44,000 genes loaded on the oligo chip.

At this time, the genes exhibiting 2.0 fold higher or less expression by amiodarone were classified according to the functions so that the genes having the functions involved in pulmonary toxicity, that is genes involved in apoptosis, lipid metabolism, cell cycle, cell proliferation, and signal transduction or transcription were selected (see Table 2 and Table 3). There have been no reports so far that those selected genes are involved in cytotoxicity in human bronchial epithelial cells according to the amiodarone treatment.

TABLE 2
Genes up-regulated by amiodarone

| | | | |
|---|---|---|---|
| | | | Ratio of |
| Accession | Gene | | intermediate |
| number | abbreviation | Gene name | value |

(a) apoptosis

| | | | |
|---|---|---|---|
| NM_006850 | IL24 | Interleukin 24 | 3.9944716 |
| NM_003820 | TNFRSF14 | Tumor necrosis factor receptor | 2.0416014 |
| | | superfamily, member | |
| | | 14 (herpesvirus | |
| | | entry mediator) | |
| BG284742 | P8 | P8 protein (candidate of | 2.4063197 |
| | | metastasis 1) | |

(b) cell cycle

| | | | |
|---|---|---|---|
| NM_014707 | HDAC9 | Histone deacetylase 9 | 2.2424666 |
| NM_003377 | VEGFB | Vascular endothelial growth | 2.0523348 |
| | | factor B | |

(c) cell proliferation

| | | | |
|---|---|---|---|
| NM_198336 | INSIG1 | Insulin induced gene 1 | 2.1351621 |
| NM_003377 | VEGFB | Vascular endothelial growth | 2.0523348 |
| | | factor B | |
| BC032783 | GPNMB | Glycoprotein (transmembrane) | 7.3201273 |
| | | nmb | |

(d) lipid metabolism

| | | | |
|---|---|---|---|
| NM_004265 | FADS2 | Fatty acid desaturase 2 | 2.0322247 |
| AK124635 | PCSK9 | Proprotein convertase | 2.2880026 |
| | | subtilisin/kexin type 9 | |
| NM_138711 | PPARG | Peroxisome proliferative | 2.0454861 |
| | | activated | |
| | | receptor, gamma | |
| NM_005063 | SCD | Stearoyl-CoA desaturase | 2.8126784 |
| | | (delta-9-desaturase) | |
| BX648997 | PPARGC1B | Peroxisome proliferative | 2.1692484 |
| | | activated | |
| | | receptor, gamma, coactivator | |
| | | 1, beta | |

(e) signal transduction

| | | | |
|---|---|---|---|
| AK124869 | | sh2 domain containg 5 | 2.7275673 |
| NM_003377 | VEGFB | Vascular endothelial growth | 2.0523348 |
| | | factor B | |
| NM_014391 | ANKRD1 | Ankyrin repeat domain | 2.3639879 |
| | | 1 (cardiac muscle) | |
| NM_138711 | PPARG | Peroxisome proliferative | 2.0454861 |
| | | activated | |
| | | receptor, gamma | |
| BM918324 | LY96 | Lymphocyte antigen 96 | 2.5142013 |
| NM_004864 | GDF15 | Growth differentiation factor | 2.8658757 |
| | | 15 | |
| NM_003820 | TNFRSF14 | Tumor necrosis factor receptor | 2.0416014 |
| | | superfamily, member | |
| | | 14 (herpesvirus entry mediator) | |
| BX648997 | PPARGC1B | Peroxisome proliferative | 2.1692484 |
| | | activated receptor, gamma, coactivator | |
| | | 1, beta | |

(f) transcription

| | | | |
|---|---|---|---|
| NM_014707 | HDAC9 | Histone deacetylase 9 | 2.2424666 |
| NM_014079 | KLF15 | Kruppel-like factor 15 | 3.2587896 |
| NM_138711 | PPARG | Peroxisome proliferative | 2.0454861 |
| | | activated receptor, gamma | |
| NM_014707 | HDAC9 | Histone deacetylase 9 | 2.2424666 |

(g) the others

| | | | |
|---|---|---|---|
| BC000054 | DHCR7 | 7-dehydrocholesterol reductase | 2.2186108 |
| AJ002231 | GNPDA1 | Glucosamine-6-phosphate | 2.5003378 |
| | | deaminase 1 | |
| NM_005559 | LAMA1 | Laminin, alpha 1 | 2.0728022 |
| AK124869 | LOC400745 | Hypothetical protein LOC149194 | 2.7275673 |
| NM_007289 | MME | Membrane metallo-endopeptidase | 2.1173943 |
| | | (neutral endopeptidase, | |
| | | enkephalinase, | |
| | | CALLA, CD10) | |
| BC013875 | MMP1 | Matrix metallopeptidase 1 | 2.7112987 |
| | | (interstitial collagenase) | |
| NM_003621 | PPFIBP2 | PTPRF interacting protein, | 2.0116139 |
| | | binding | |
| | | Protein 2 (liprin beta 2) | |
| BC033883 | RBP7 | Retinol binding protein 7, | 2.0115712 |
| | | cellular | |
| AL137502 | RRAGD | Ras-related GTP binding D | 4.0888656 |
| NM_003627 | SLC43A1 | Solute carrier family 43, | 2.0899673 |
| | | member 1 | |
| AL834346 | STXBP6 | Syntaxin binding protein 6 | 2.1482119 |
| | | (amisyn) | |
| NM_033035 | TSLP | Thymic stromal lymphopoietin | 2.0715997 |

(h) Biological process unknown

| | | | |
|---|---|---|---|
| BC050651 | BEX2 | Brain expressed X-linked 2 | 2.3649894 |
| AK128526 | C9orf58 | Chromosome 9 open reading | 2.0017072 |
| | | frame 58 | |
| XM_044178 | KIAA1211 | KIAA1211 protein | 2.1775467 |
| AK090454 | LOC150946 | Family with sequence | 3.2247295 |
| | | similarity 59, | |
| | | member B | |
| AK094730 | LOC283454 | Hypothetical protein LOC283454 | 2.3452545 |
| BX537968 | LOC51149 | Hypothetical LOC51149 | 2.1573757 |
| CR617492 | MGC15887 | Family with sequence | 2.1979278 |
| | | similarity 89, | |
| | | member A | |
| XM_350880 | PPM1H | Protein phosphatase 1H (PP2C | 2.536865 |
| | | domain containing) | |
| NM_012449 | STEAP | Six transmembrane epithelial | 2.0866588 |
| | | antigen of the prostate 1 | |
| AL832142 | TM7SF1 | Transmembrane 7 superfamily | 2.0007024 |
| | | member 1 (upregulated in kidney) | |
| ENST00000313481 | Unknown | | 2.0008635 |
| AW665665 | Unknown | Transcribed locus, strongly | 2.0599298 |
| | | similar to XP_509406.1 | |
| | | PREDICTED: similar to | |
| | | hypothetical protein | |
| | | FLJ14627 [Pan | |
| | | troglodytes] | |

TABLE 3
Genes down-regulated by amiodarone

| | | | |
|---|---|---|---|
| | | | Ratio of |
| | Gene | | intermediate |
| Accession number | abbreviation | Gene name | value |

(a) cell cycle

| | | | |
|---|---|---|---|
| AB209179 | PLK1 | Polo-like kinase 1 | 0.4237926 |
| | | (Drosophila) | |
| AY367065 | ASPM | Asp (abnormal spindle)-like, | 0.4140736 |
| | | microcephaly | |
| | | associated (Drosophila) | |
| NM_031966 | CCNB1 | Cyclin B1 | 0.4422999 |
| NM_001813 | CENPE | Centromere protein E, 312 kDa | 0.4713322 |
| BC043371 | GFI1B | Growth factor independent 1B | 0.4864506 |
| | | (potential regulator of | |
| | | CDKN1A, translocated in CML) | |

(b) cell proliferation

| | | | |
|---|---|---|---|
| AB209179 | PLK1 | Polo-like kinase 1 | 0.4237926 |
| | | (Drosophila) | |
| BC043371 | GFI1B | Growth factor independent 1B | 0.4864506 |
| | | (potential | |
| | | translocated in CML) | |

(c) signal transduction

| | | | |
|---|---|---|---|
| BC065304 | DEPDC1 | DEP domain containing 1 | 0.4510642 |

(d) transcription

| | | | |
|---|---|---|---|
| NM_001964 | EGR1 | Early growth response 1 | 0.4390462 |
| BC043371 | GFI1B | Growth factor independent 1B | 0.4864506 |
| | | (potential regulator of | |
| | | CDKN1A, | |
| | | translocated in CML) | |
| NM_005933 | MLL | Myeloid/lymphoid or mixed- | 0.4820172 |
| | | lineage | |
| | | leukemia (trithorax homolog, | |
| | | Drosophila) | |
| NM_012333 | MYCBP | C-myc binding protein | 0.4922388 |
| AB095943 | SHPRH | SNF2 histone linker PHD RING | 0.4682101 |
| | | helicase | |
| NM_016267 | VGLL1 | Vestigial like 1 (Drosophila) | 0.442002 |

(e) the others

| | | | |
|---|---|---|---|
| NM_170589 | AF15Q14 | Cancer susceptibility | 0.4207353 |
| | | candidate 5 | |
| NM_000046 | ARSB | Arylsulfatase B | 0.3982376 |
| BC036307 | CNN1 | Calponin 1, basic, smooth | 0.475635 |
| | | muscle | |
| AB209653 | DHRS2 | Dehydrogenase/reductase (SDR | 0.4920274 |
| | | family) | |
| | | member 2 | |
| AK122613 | FLJ16025 | ATPase type 13A5 | 0.4491728 |
| NM_005319 | HIST1H1C | Histone 1, H1c | 0.4988284 |
| NM_003512 | HIST1H2AC | Histone 1, H2ac | 0.4876834 |
| NM_080593 | HIST1H2BK | Histone 1, H2bk | 0.3641802 |
| BC069193 | HIST2H2BE | Histone 2, H2be | 0.3240014 |
| AF032862 | HMMR | Hyaluronan-mediated motility | 0.492606 |
| | | receptor | |
| | | (RHAMM) | |
| AY791349 | KIF18A | Kinesin family member 18A | 0.4719817 |
| AK025790 | KIF20A | Kinesin family member 20A | 0.3397137 |
| NM_001620 | MGC5395 | AHNAK nucleoprotein | 0.2681892 |
| | | (desmoyokin) | |
| NM_012333 | MYCBP | C-myc binding protein | 0.4922388 |
| NM_006472 | TXNIP | Thioredoxin interacting | 0.3991791 |
| | | protein | |
| DQ097177 | UREB1 | HECT, UBA and WWE domain | 0.3849775 |
| | | containing 1 | |
| NM_006904 | PRKDC | Protein kinase, DNA-activated | 0.4833296 |
| | | catalytic polypeptide | |

(f) Biological process unknown

| | | | |
|---|---|---|---|
| BC053619 | ARRDC3 | Arrestin domain containing 3 | 0.4160297 |
| NM_052876 | BTBD14B | BTB (POZ) domain containing | 0.4198905 |
| | | 14B | |
| M28016 | cytochrome b | Human mitochondrial | 0.4492316 |
| | | cytochrome b | |
| | | gene, partial cds. | |
| BC082232 | HIST1H2BG | Histone 1, H2bg | 0.4080795 |
| BC101655 | HIST1H2BI | Histone 1, H2bi | 0.4471735 |
| BM752802 | HIST1H2BM | Histone 1, H2bm | 0.3949135 |
| BX647290 | HIST1H2BO | Histone 1, H2bo | 0.433098 |
| BC029844 | LOC256021 | Hypothetical protein | 0.4326738 |
| | | LOC256021 | |
| AF001540 | MALAT-1 | metastasis associated | 0.3449582 |
| | | lunadenocarcinoma transcript 1 | |
| | | (non-coding RNA) | |
| AJ002535 | OBSCN | Obscurin, cytoskeletal | 0.3870984 |
| | | calmodulin and | |
| | | titin-interacting RhoGEF | |
| BC050630 | RAB3-GAP150 | RAB3 GTPase activating | 0.4663124 |
| | | protein | |
| | | subunit 2 (non-catalytic) | |
| AB016092 | SRRM2 | Serine/arginine repetitive | 0.3916611 |
| | | matrix 2 | |
| CR600908 | | full-length cDNA clone | 0.4399871 |
| | | CS0DL005YJ22 | |
| | | of B cells (Ramos cell line) | |
| | | Cot | |
| | | 25-normalized of Homo sapiens | |
| | | (human). | |
| THC2428713 | | AF136551 cytochrome b {Sus | 0.4469303 |
| | | scrofa;}, | |
| | | partial (48%) [THC2428713] | |
| BE620675 | | Transcribed locus, moderately | 0.4453037 |
| | | similar | |
| | | to NP_536855.1 cytochrome b | |
| | | [Homo sapiens] | |
| AY358369 | | PRO333; Homo sapiens clone | 0.4609807 |
| | | DNA41374 | |
| | | SIGLEC5 (UNQ294) mRNA, | |
| | | partial cds. | |
| BE300829 | | Transcribed locus, moderately | 0.3894044 |
| | | similar | |
| | | to NP_005021.2 polo-like | |
| | | kinase; polo | |
| | | (Drosophia)-like kinase; polo-like | |
| | | kinase (Drosophila) [Homo | |
| | | sapiens] | |

### Example 3

### Quantification by Real Time RT-PCR (Reverse Transcriptase Polymerase Chain Reaction)

Among genes apt to be up-regulated or down-regulated by amiodarone, confirmed in Example 2, 12 genes involved in lipid metabolism, cell cycle and signal transduction, as pulmonary toxicity related mechanisms, were selected. The genes are Genebank Accession No: AB209179 [Polo-like kinase 1 (Drosophila)], Genebank Accession No: NM_{—}031966 (Cyclin B1), Genebank Accession No: BC043371 [Growth factor independent 1B (potential regulator of CDKN1A, translocated in CML)], Genebank Accession No: NM_{—}004265 (Fatty acid denaturase 2), Genebank Accession No: NM_{—}138711 (Peroxisome proliferative activated receptor, gamma), Genebank Accession No: NM_{—}005063 [Stearoyl-CoA denaturase (delta-9-denaturase)], Genebank Accession No: AK124869 (sh2 domain containing 5), Genebank Accession No: NM_{—}014391 [Ankyrin repeat domain 1 (cardiac muscle)], Genebank Accession No: BM918324 (Lymphocyte antigen 96), Genebank Accession No: NM_{—}004864 (Growth differentiation factor 15), Genebank Accession No: NM_{—}003820 [Tumor necrosis factor receptor superfamily, member 14 (herpes virus entry mediator)] and Genebank Accession No: BC065304 (DEP domain containing 1).

To investigate and quantify the expressions of those genes, quantitative real time RT-PCR was performed using My IQ Real-time PCR (Bio-rad, USA). Particularly, reverse transcription was performed using oligo dT primer and Superscript kit (Omniscipt™ kit, Qiagen, Co., USA) to synthesize cDNA. 0.2 μl of the cDNA, 3.8 μl of water, 0.5 μl of sense primer, 0.5 AS of antisense primer, and 5 μl of SYBR Green I staining supermix (Bio-rad, USA) were mixed, and the mixture was placed in PCR tube, followed by RT-PCR in My IQ real time PCR machine as follows: step 1, at 95□ for 3 minutes; step 2 (repeated 45 times), step 2-1, at 95□ for 10 seconds, step 2-2, at 55-65□ for 45 seconds; step 3, at 95□ for 1 minute; step 4 at 55□ for 1 minute; and step 5 (repeated 80 times) at 55□ for 10 seconds. To quantify PCR product, SYBR Green I (Bio-rad, USA) staining was performed. SYBR Green I staining is the staining method based on binding to double stranded DNA, so that as double stranded DNA increases during PCR, fluorescence intensity increases. The target gene used for PCR and primers for endogenous GAPDH were added to SYBR Green master mix, followed by PCR. Then, primer optimization was performed to select optimum concentration. The synthesized cDNA was mixed with each primer (Table 4), to which the SYBR Green master mix was added, followed by PCR. Quantification was performed using quantitative software (see Table 5).

TABLE 4
Primer sequences

| Accees- | | | |
|---|---|---|---|
| sion | | PCR primer sequence | |
| Number | Gene name | (5′->3′) | |
| AB209179 | Polo-like kinase | sense | CTCAACACGCCTC |
| | 1 (Drosophila) | (sequence | ATCCTC |
| | | number 1) | |
| | | antisense | GTGCTCGCTCATG |
| | | (sequence | TAATTGC |
| | | number 2) | |
| NM_031966 | Cyclin B1 | sense | TCTGGATAATGGT |
| | | (sequence | GAATGGACA |
| | | number 3) | |
| | | antisense | CGATGTGGCATAC |
| | | (sequence | TTGTTCTTG |
| | | number 4) | |
| BC043371 | Growth factor | sense | TTCCTGGTGAAGA |
| | independent | (sequence | GCAAGAAGGCT |
| | 1B (potential | number 5) | |
| | regulator of | antisense | TCCAGGCACTGGT |
| | CDKN1A, | (sequence | TTGGGAATAGA |
| | translocated in | number 6) | |
| | CML) | | |
| NM_004265 | Fatty acid | sense | TGGATGGAACAGC |
| | desaturase 2 | (sequence | TAAGGCCAAGA |
| | | number 7) | |
| | | antisense | CTGTGGTTTGCAG |
| | | (sequence | CCAGATGGTTT |
| | | number 8) | |
| NM_138711 | Peroxisome | sense | CACAAGAACAGAT |
| | proliferative | (sequence | CCAGTGGTTGCAG |
| | activated | number 9) | |
| | receptor, gamma | antisense | AATAATAAGGTGG |
| | | (sequence | AGATGCAGGCTCC |
| | | number 10) | |
| NM_005063 | Stearoyl-CoA | sense | AACTTGATACGTC |
| | desaturase | (sequence | CGTGTGTCCCA |
| | (delta-9- | number 11) | |
| | desaturase) | antisense | CTGTATGTTTCCG |
| | | (sequence | TGGCAATGCGT |
| | | number 12) | |
| AK124869 | sh2 domain | sense | AGACCTGGTCATT |
| | containg 5 | (sequence | GGTCCAGACTT |
| | | number 13) | |
| | | antisense | AACATGGCCCTGA |
| | | (sequence | TAGCTTCTCCA |
| | | number 14) | |
| NM_014391 | Ankyrin repeat | sense | AAGCGAGAAACAA |
| | domain 1 | (sequence | CGAGAGGCAGA |
| | (cardiac muscle) | number 15) | |
| | | antisense | AGAAACGTAGGCA |
| | | (sequence | CATCCACAGGT |
| | | number 16) | |
| BM918324 | Lymphocyte | sense | AGCTCTGAAGGGA |
| | antigen 96 | (sequence | GAGACTGTGAA |
| | | number 17) | |
| | | antisense | GGTGTAGGATGAC |
| | | (sequence | AAACTCCAAGC |
| | | number 18) | |
| NM_004864 | Growth | sense | AAGAACTCAGGAC |
| | differentiation | (sequence | GGTGAATGGCT |
| | factor 15 | number 19) | |
| | | antisense | TTTCCGCAACTCT |
| | | (sequence | CGGAATCTGGA |
| | | number 20) | |
| NM_003820 | Tumor necrosis | sense | AGGAATGTCAGCA |
| | factor receptor | (sequence | CCAGACCAAGT |
| | superfamily, | number 21) | |
| | member | antisense | GGCCAACTGTGGA |
| | 14 (herpesvirus | (sequence | GCAAACAATGA |
| | entry mediator) | number 22) | |
| BC065304 | DEP domain | sense | GCCACCAAGCTGT |
| | containing 1 | (sequence | GGAATGAAGTT |
| | | number 23) | |
| | | antisense | ATCCACTGCTTCT |
| | | (sequence | CCTGCTGTGAA |
| | | number 24) | |

TABLE 5

| | | Real time | cDNA |
|---|---|---|---|
| | | PCR | microarray |
| Accession | | (relative | (Cy3/Cy5 |
| Number | Gene name | ratio) | ratio) |
| AB209179 | Polo-like kinase 1 | 0.4238 | 0.349492 |
| | (Drosophila) | | |
| NM_031966 | Cyclin B1 | 0.4423 | 0.480742 |
| BC043371 | Growth factor independent 1B | 0.4865 | 0.382447 |
| | (potential regulator of | | |
| | CDKN1A, translocated in CML) | | |
| NM_004265 | Fatty acid desaturase 2 | 2.0322 | 3.498331 |
| NM_138711 | Peroxisome proliferative | 2.0455 | 4.037139 |
| | activated receptor, gamma | | |
| NM_005063 | Stearoyl-CoA desaturase | 2.8127 | 3.990769 |
| | (delta-9-desaturase) | | |
| AK124869 | sh2 domain containg 5 | 2.7276 | 3.792984 |
| NM_014391 | Ankyrin repeat domain 1 | 2.364 | 5.464161 |
| | (cardiac muscle) | | |
| BM918324 | Lymphocyte antigen 96 | 2.5142 | 5.426417 |
| NM_004864 | Growth differentiation | 2.8659 | 3.317278 |
| | factor 15 | | |
| NM_003820 | Tumor necrosis factor | 2.0416 | 4.8121 |
| | receptor superfamily, member | | |
| | 14 (herpesvirus entry | | |
| | mediator) | | |
| BC065304 | DEP domain containing 1 | 0.4511 | 0.403321 |

As a result, 8 up-regulated genes and 4 down-regulated genes were identified, and these expression patterns were consistent with the result of oligo-microarray examining the gene expressions by the drugs inducing pulmonary toxicity.

### INDUSTRIAL APPLICABILITY

The marker gene of the present invention can be effectively used for monitoring and identifying drugs or chemical having high risk of inducing pulmonary toxicity and can be used as an effective tool for examining the mechanism of amiodarone which causes pulmonary toxicity and side effects.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for screening a drug candidate inducing pulmonary toxicity comprising the following steps:
1) treating human bronchial epithelial cells with a sample compound or composition as an experimental group;
2) comparing expression level of a group of genes whose expression is altered by treatment of amiodarone between the experimental group of step 1) and a control group which are human bronchial epithelial cells untreated with the sample compound or composition; and
3) selecting the compound or composition which alters the expression level of the group of the genes significantly comparing to the control group.

2. The method according to claim 1, wherein the human bronchial epithelial cells of step 1) are BEAS-2B cells, human bronchus cells or human lung cells.

3. The method according to claim 1, wherein the group of genes is up-regulated when exposed to amiodarone.

4. The method according to claim 1, wherein the group of genes is down-regulated when exposed to amiodarone.

5. The method according to claim 3, wherein the group of genes contains at least one gene selected from the group consisting of:
Genebank Accession Number: NM_{—}014391 [Ankyrin repeat domain 1 (cardiac muscle)], Genebank Accession Number: BC050651 (Brain expressed X-linked 2), Genebank Accession Number: AK128526 (Chromosome 9 open reading frame 58), Genebank Accession Number: BC000054 (7-dehydrocholesterol reductase), Genebank Accession Number: NM_{—}004265 (Fatty acid desaturase), Genebank Accession Number: NM_{—}004864 (Growth differentiation factor 15), Genebank Accession Number: AJ002231 (Glucosamine-6-phosphate deaminase 1), Genebank Accession Number: BC032783 (Glycoprotein (transmembrane) nmb), Genebank Accession Number: NM_{—}014707 (Histone deacetylase 9), Genebank Accession Number: T92260 (Transcribed locus), Genebank Accession Number: BE614991 [Transcribed locus, strongly similar to XP_{—}514491.1 PREDICTED: similar to hypothetical protein (Pan troglodytes)], Genebank Accession Number: BE614991 [Transcribed locus, strongly similar to XP_{—}514491.1 PREDICTED: similar to hypothetical protein (Pan troglodytes)], Genebank Accession Number: AF026246, Genebank Accession Number: NM_{—}006850 (Interleukin 24), Genebank Accession Number: NM_{—}198336 (Insulin induced gene 1), Genebank Accession Number: XM_{—}044178 (KIAA1211 protein), Genebank Accession Number: NM_{—}014079 (Kruppel-like factor 15), Genebank Accession Number: NM_{—}005559 (Laminin, alpha 1), Genebank Accession Number: AK090454 (Family with sequence similarity 59, member B), Genebank Accession Number: AK094730 (Hypothetical protein LOC283454), Genebank Accession Number: AK124869 (Hypothetical protein LOC149194), Genebank Accession Number: BX537968 (Hypothetical LOC51149), Genebank Accession Number: BM918324 (Lymphocyte antigen 96), Genebank Accession Number: CR617492 (Family with sequence similarity 89, member A), Genebank Accession Number: NM_{—}007289 [Membrane metallo-endopeptidase (neutral endopeptidase, enkephalinase, CALLA, CD10)], Genebank Accession Number: BC013875 [Matrix metallopeptidase 1 (interstitial collagenase)], Genebank Accession Number: BG284742 [P8 protein (candidate of metastasis 1)], Genebank Accession Number: AK124635 (Proprotein convertase subtilisin/kexin type 9), Genebank Accession Number: NM_{—}138711 (Peroxisome proliferative activated receptor, gamma), Genebank Accession Number: BX648997 (Peroxisome proliferative activated receptor, gamma, coactivator 1, beta), Genebank Accession Number: NM_{—}003621 [PTPRF interacting protein, binding protein 2 (liprin beta 2)], Genebank Accession Number: XM_{—}350880 [Protein phosphatase 1H (PP2C domain containing)], Genebank Accession Number: BC033883 (Retinol binding protein 7, cellular), Genebank Accession Number: AL137502 (Ras-related GTP binding D), Genebank Accession Number: NM_{—}005063 [Stearoyl-CoA desaturase (delta-9-desaturase)], Genebank Accession Number: NM_{—}003627 (Solute carrier family 43, member 1), Genebank Accession Number: NM_{—}012449 (Six transmembrane epithelial antigen of the prostate 1), Genebank Accession Number: AL834346 [Syntaxin binding protein 6 (amisyn)], Genebank Accession Number: AL832142 [Transmembrane 7 superfamily member 1 (upregulated in kidney)], Genebank Accession Number: NM_{—}003820 [Tumor necrosis factor receptor superfamily, member 14 (herpesvirus entry mediator)], Genebank Accession Number: NM_{—}033035 (Thymic stromal lymphopoietin), Genebank Accession Number: AW665665 [Transcribed locus, strongly similar to XP_{—}509406.1 PREDICTED: similar to hypothetical protein FLJ14627 (Pan troglodytes)], Genebank Accession Number: NM_{—}003377 (Vascular endothelial growth factor B), EnsEMBL Accession Number: ENST00000313481.

6. The method according to claim 4, wherein the group of genes contains at least one gene selected from the group consisting of:
Genebank Accession Number: NM_{—}170589 (Cancer susceptibility candidate 5), Genebank Accession Number: BC053619 (Arrestin domain containing 3), Genebank Accession Number: NM_{—}000046 (Arylsulfatase B), Genebank Accession Number: AY367065 [Asp (abnormal spindle)-like, microcephaly associated (Drosophila)], Genebank Accession Number: NM_{—}052876 [BTB (POZ) domain containing 14B], Genebank Accession Number: NM_{—}031966 (Cyclin B1), Genebank Accession Number: NM_{—}001813 (Centromere protein E, 312 kDa), Genebank Accession Number: BC036307 (Calponin 1, basic, smooth muscle), Genebank Accession Number: M28016 (Human mitochondrial cytochrome b gene, partial cds.), Genebank Accession Number: BC065304 (DEP domain containing 1), Genebank Accession Number: AB209653 [Dehydrogenase/reductase (SDR family) member 2], Genebank Accession Number: NM_{—}001964 (Early growth response 1), Genebank Accession Number: AK122613 (ATPase type 13A5), Genebank Accession Number: CR600908 [full-length cDNA clone CS0DL005YJ22 of B cells (Ramos cell line) Cot 25-normalized of Homo sapiens(human).], Genebank Accession Number: BC043371 [Growth factor independent 1B (potential regulator of CDKN1A, translocated in CML)], Genebank Accession Number: NM_{—}016548 (Golgi phosphoprotein 2), Genebank Accession Number: NM_{—}005319 (Histone 1, H1c), Genebank Accession Number: NM_{—}003512 (Histone 1, H2ac), Genebank Accession Number: BC082232 (Histone 1, H2bg), Genebank Accession Number: BC101655 (Histone 1, H2bi), Genebank Accession Number: NM_{—}080593 (Histone 1, H2bk), Genebank Accession Number: BM752802 (Histone 1, H2bm), Genebank Accession Number: BX647290 (Histone 1, H2bo), Genebank Accession Number: BC069193 (Histone 2, H2be), Genebank Accession Number: AF032862 [Hyaluronan-mediated motility receptor (RHAMM)], Genebank Accession Number: BE620675 [Transcribed locus, moderately similar to NP_{—}536855.1 cytochrome b (Homo sapiens)], Genebank Accession Number: AY358369 [PRO333; Homo sapiens clone DNA41374 SIGLEC5 (UNQ294) mRNA, partial cds.], Genebank Accession Number: BE300829 [Transcribed locus, moderately similar to NP_{—}005021.2 polo-like kinase; polo (Drosophila)-like kinase; polo-like kinase (Drosophila) (Homo sapiens)], Genebank Accession Number: AY791349 (Kinesin family member 18A), Genebank Accession Number: AK025790 (Kinesin family member 20A), Genebank Accession Number: BC029844 (Hypothetical protein LOC256021), Genebank Accession Number: AF001540 [metastasis associated lung adenocarcinoma transcript 1 (non-coding RNA)], Genebank Accession Number: NM_{—}001620 [AHNAK nucleoprotein (desmoyokin)], Genebank Accession Number: NM_{—}00593 [Myeloid/lymphoid or mixed-lineage leukemia (trithorax homolog, Drosophila)], Genebank Accession Number: NM_{—}012333 (C-myc binding protein), Genebank Accession Number: AJ002535 (Obscurin, cytoskeletal calmodulin and titin-interacting RhoGEF), Genebank Accession Number: AB209179 [Polo-like kinase 1 (Drosophila)], Genebank Accession Number: NM_{—}006904 (Protein kinase, DNA-activated, catalytic polypeptide), Genebank Accession Number: BC050630 [RAB3 GTPase activating protein subunit 2 (non-catalytic)], Genebank Accession Number: AB095943 (SNF2 histone linker PHD RING helicase), Genebank Accession Number: AB016092 (Serine/arginine repetitive matrix 2), Genebank Accession Number: NM_{—}006472 (Thioredoxin interacting protein), Genebank Accession Number: DQ097177 (HECT, UBA and WWE domain containing 1), Genebank Accession Number: NM_{—}016267 [Vestigial like 1 (Drosophila)], TIGR Accession Number: THC2428713.

7. The method according to claim 1, wherein the expression level of step 2) is measured as mRNA level or protein level.

8. The method according to claim 7, wherein the mRNA level is measured by oligonucleotide or polynucleotide microarray, or RT-PCR.

9. The method according to claim 7, wherein the protein level is measured by protein microarray or ELISA.

10. A method for screening a drug candidate inducing pulmonary toxicity comprising the following steps:
1) treating human bronchial epithelial cells with a sample compound or composition as an experimental group;
2) extracting RNAs from the experimental group of step 1) and from the control group which are human bronchial epithelial cells untreated with the sample compound or composition;
3) converting RNAs extracted from the experimental group and the control group of step 2) into cDNA and labeling them with different fluorescent materials;
4) hybridizing cDNAs labeled with different fluorescent materials of step 3) with a DNA microarray containing a group of genes whose expression is altered by treatment of amiodarone;
5) analyzing the reacted DNA microarray chip of step 4);
6) comparing the expression level of a group of genes whose expression is altered by treatment of amiodarone between the experimental group and a control group based on the data analyzed in step 5); and
7) selecting the compound or composition which alters the expression level of the group of the genes significantly comparing to the control group.

11. The method according to claim 10, wherein the human bronchial epithelial cells of step 1) are BEAS-2B cells, human bronchus cells or human lung cells.

12. The method according to claim 10, wherein the group of genes is up-regulated when exposed to amiodarone.

13. The method according to claim 10, wherein the group of genes is down-regulated when exposed to amiodarone.

14. The method according to claim 12, wherein the group of genes contains at least one gene selected from the group consisting of:
Genebank Accession Number: NM_{—}014391 [Ankyrin repeat domain 1 (cardiac muscle)], Genebank Accession Number: BC050651 (Brain expressed X-linked 2), Genebank Accession Number: AK128526 (Chromosome 9 open reading frame 58), Genebank Accession Number: BC000054 (7-dehydrocholesterol reductase), Genebank Accession Number: NM_{—}004265 (Fatty acid desaturase), Genebank Accession Number: NM_{—}004864 (Growth differentiation factor 15), Genebank Accession Number: AJ002231 (Glucosamine-6-phosphate deaminase 1), Genebank Accession Number: BC032783 (Glycoprotein (transmembrane) nmb), Genebank Accession Number: NM_{—}014707 (Histone deacetylase 9), Genebank Accession Number: T92260 (Transcribed locus), Genebank Accession Number: BE614991 [Transcribed locus, strongly similar to XP_{—}514491.1 PREDICTED: similar to hypothetical protein (Pan troglodytes)], Genebank Accession Number: BE614991 [Transcribed locus, strongly similar to XP_{—}514491.1 PREDICTED: similar to hypothetical protein (Pan troglodytes)], Genebank Accession Number: AF026246, Genebank Accession Number: NM_{—}006850 (Interleukin 24), Genebank Accession Number: NM_{—}198336 (Insulin induced gene 1), Genebank Accession Number: XM_{—}044178 (KIAA1211 protein), Genebank Accession Number: NM_{—}014079 (Kruppel-like factor 15), Genebank Accession Number: NM_{—}005559 (Laminin, alpha 1), Genebank Accession Number: AK090454 (Family with sequence similarity 59, member B), Genebank Accession Number: AK094730 (Hypothetical protein LOC283454), Genebank Accession Number: AK124869 (Hypothetical protein LOC149194), Genebank Accession Number: BX537968 (Hypothetical LOC51149), Genebank Accession Number: BM918324 (Lymphocyte antigen 96), Genebank Accession Number: CR617492 (Family with sequence similarity 89, member A), Genebank Accession Number: NM_{—}007289 [Membrane metallo-endopeptidase (neutral endopeptidase, enkephalinase, CALLA, CD10)], Genebank Accession Number: BC013875 [Matrix metallopeptidase 1 (interstitial collagenase)], Genebank Accession Number: BG284742 [P8 protein (candidate of metastasis 1)], Genebank Accession Number: AK124635 (Proprotein convertase subtilisin/kexin type 9), Genebank Accession Number: NM_{—}138711 (Peroxisome proliferative activated receptor, gamma), Genebank Accession Number: BX648997 (Peroxisome proliferative activated receptor, gamma, coactivator 1, beta), Genebank Accession Number: NM_{—}003621 [PTPRF interacting protein, binding protein 2 (liprin beta 2)], Genebank Accession Number: XM_{—}350880 [Protein phosphatase 1H (PP2C domain containing)], Genebank Accession Number: BC033883 (Retinol binding protein 7, cellular), Genebank Accession Number: AL137502 (Ras-related GTP binding D), Genebank Accession Number: NM_{—}005063 [Stearoyl-CoA desaturase (delta-9-desaturase)], Genebank Accession Number: NM_{—}003627 (Solute carrier family 43, member 1), Genebank Accession Number: NM_{—}012449 (Six transmembrane epithelial antigen of the prostate 1), Genebank Accession Number: AL834346 [Syntaxin binding protein 6 (amisyn)], Genebank Accession Number: AL832142 [Transmembrane 7 superfamily member 1 (upregulated in kidney)], Genebank Accession Number: NM_{—}003820 [Tumor necrosis factor receptor superfamily, member 14 (herpesvirus entry mediator)], Genebank Accession Number: NM_{—}033035 (Thymic stromal lymphopoietin), Genebank Accession Number: AW665665 [Transcribed locus, strongly similar to XP_{—}509406.1 PREDICTED: similar to hypothetical protein FLJ14627 (Pan troglodytes)], Genebank Accession Number: NM_{—}003377 (Vascular endothelial growth factor B), EnsEMBL Accession Number: ENST00000313481.

15. The method according to claim 13, wherein the group of genes contains at least one gene selected from the group consisting of:
Genebank Accession Number: NM_{—}170589 (Cancer susceptibility candidate 5), Genebank Accession Number: BC053619 (Arrestin domain containing 3), Genebank Accession Number: NM_{—}000046 (Arylsulfatase B), Genebank Accession Number: AY367065 [Asp (abnormal spindle)-like, microcephaly associated (Drosophila)], Genebank Accession Number: NM_{—}052876 [BTB (POZ) domain containing 14B], Genebank Accession Number: NM_{—}031966 (Cyclin B1), Genebank Accession Number: NM_{—}001813 (Centromere protein E, 312 kDa), Genebank Accession Number: BC036307 (Calponin 1, basic, smooth muscle), Genebank Accession Number: M28016 (Human mitochondrial cytochrome b gene, partial cds.), Genebank Accession Number: BC065304 (DEP domain containing 1), Genebank Accession Number: AB209653 [Dehydrogenase/reductase (SDR family) member 2], Genebank Accession Number: NM_{—}001964 (Early growth response 1), Genebank Accession Number: AK122613 (ATPase type 13A5), Genebank Accession Number: CR600908 [full-length cDNA clone CS0DL005YJ22 of B cells (Ramos cell line) Cot 25-normalized of Homo sapiens(human).], Genebank Accession Number: BC043371 [Growth factor independent 1B (potential regulator of CDKN1A, translocated in CML)], Genebank Accession Number: NM_{—}016548 (Golgi phosphoprotein 2), Genebank Accession Number: NM_{—}005319 (Histone 1, H1c), Genebank Accession Number: NM_{—}003512 (Histone 1, H2ac), Genebank Accession Number: BC082232 (Histone 1, H2bg), Genebank Accession Number: BC101655 (Histone 1, H2bi), Genebank Accession Number: NM_{—}080593 (Histone 1, H2bk), Genebank Accession Number: BM752802 (Histone 1, H2bm), Genebank Accession Number: BX647290 (Histone 1, H2bo), Genebank Accession Number: BC069193 (Histone 2, H2be), Genebank Accession Number: AF032862 [Hyaluronan-mediated motility receptor (RHAMM)], Genebank Accession Number: BE620675 [Transcribed locus, moderately similar to NP_{—}536855.1 cytochrome b (Homo sapiens)], Genebank Accession Number: AY358369 [PRO333; Homo sapiens clone DNA41374 SIGLEC5 (UNQ294) mRNA, partial cds.], Genebank Accession Number: BE300829 [Transcribed locus, moderately similar to NP_{—}005021.2 polo-like kinase; polo (Drosophila)-like kinase; polo-like kinase (Drosophila)(Homo sapiens)], Genebank Accession Number: AY791349 (Kinesin family member 18A), Genebank Accession Number: AK025790 (Kinesin family member 20A), Genebank Accession Number: BC029844 (Hypothetical protein LOC256021), Genebank Accession Number: AF001540 [metastasis associated lung adenocarcinoma transcript 1 (non-coding RNA)], Genebank Accession Number: NM_{—}001620 [AHNAK nucleoprotein (desmoyokin)], Genebank Accession Number: NM_{—}00593 [Myeloid/lymphoid or mixed-lineage leukemia (trithorax homolog, Drosophila)], Genebank Accession Number: NM_{—}012333 (C-myc binding protein), Genebank Accession Number: AJ002535 (Obscurin, cytoskeletal calmodulin and titin-interacting RhoGEF), Genebank Accession Number: AB209179 [Polo-like kinase 1 (Drosophila)], Genebank Accession Number: NM_{—}006904 (Protein kinase, DNA-activated, catalytic polypeptide), Genebank Accession Number: BC050630 [RAB3 GTPase activating protein subunit 2 (non-catalytic)], Genebank Accession Number: AB095943 (SNF2 histone linker PHD RING helicase), Genebank Accession Number: AB016092 (Serine/arginine repetitive matrix 2), Genebank Accession Number: NM_{—}006472 (Thioredoxin interacting protein), Genebank Accession Number: DQ097177 (HECT, UBA and WWE domain containing 1), Genebank Accession Number: NM_{—}016267 [Vestigial like 1 (Drosophila)], TIGR Accession Number: THC2428713.

16. The method according to claim 10, wherein the fluorescent material of step 3) is selected from the group consisting of Cy3, Cy5, poly L-lysine-fluorescein isothiocyanate (FITC), rhodamine-B-isothiocyanate (RITC) and rhodamine.

17. The method according to claim 10, wherein the DNA microarray of step 4) is oligonucleotide or polynucleotide microarray.

18. A method for screening a drug candidate inducing pulmonary toxicity comprising the following steps:
1) treating human bronchial epithelial cells with a sample compound or composition as an experimental group;
2) extracting RNAs from the experimental group of step 1) and from the control group which are human bronchial epithelial cells untreated with the sample compound or composition;
3) performing real time RT-PCR with the RNAs of step 2) using primers corresponding to a group of genes whose expression is altered by treatment of amiodarone and capable of amplifying the group of genes;
4) comparing expression level of the group of genes between the experimental group and a control group; and
5) selecting the compound or composition which alters the expression level of the group of the genes significantly comparing to the control group.

19. The method according to claim 18, wherein the human bronchial epithelial cells of step 1) are BEAS-2B cells, human bronchus cells or human lung cells.

20. The method according to claim 18, wherein the group of genes is up-regulated when exposed to amiodarone.

21. The method according to claim 18, wherein the group of genes is down-regulated when exposed to amiodarone.

22. The method according to claim 20, wherein the group of genes contains at least one gene selected from the group consisting of:
Genebank Accession Number: NM_{—}014391 [Ankyrin repeat domain 1 (cardiac muscle)], Genebank Accession Number: BC050651 (Brain expressed X-linked 2), Genebank Accession Number: AK128526 (Chromosome 9 open reading frame 58), Genebank Accession Number: BC000054 (7-dehydrocholesterol reductase), Genebank Accession Number: NM_{—}004265 (Fatty acid desaturase), Genebank Accession Number: NM_{—}004864 (Growth differentiation factor 15), Genebank Accession Number: AJ002231 (Glucosamine-6-phosphate deaminase 1), Genebank Accession Number: BC032783 (Glycoprotein (transmembrane) nmb), Genebank Accession Number: NM_{—}014707 (Histone deacetylase 9), Genebank Accession Number: T92260 (Transcribed locus), Genebank Accession Number: BE614991 [Transcribed locus, strongly similar to XP_{—}514491.1 PREDICTED: similar to hypothetical protein (Pan troglodytes)], Genebank Accession Number: BE614991 [Transcribed locus, strongly similar to XP_{—}514491.1 PREDICTED: similar to hypothetical protein (Pan troglodytes)], Genebank Accession Number: AF026246, Genebank Accession Number: NM_{—}006850 (Interleukin 24), Genebank Accession Number: NM_{—}198336 (Insulin induced gene 1), Genebank Accession Number: XM_{—}044178 (KIAA1211 protein), Genebank Accession Number: NM_{—}014079 (Kruppel-like factor 15), Genebank Accession Number: NM_{—}005559 (Laminin, alpha 1), Genebank Accession Number: AK090454 (Family with sequence similarity 59, member B), Genebank Accession Number: AK094730 (Hypothetical protein LOC283454), Genebank Accession Number: AK124869 (Hypothetical protein LOC149194), Genebank Accession Number: BX537968 (Hypothetical LOC51149), Genebank Accession Number: BM918324 (Lymphocyte antigen 96), Genebank Accession Number: CR617492 (Family with sequence similarity 89, member A), Genebank Accession Number: NM_{—}007289 [Membrane metallo-endopeptidase (neutral endopeptidase, enkephalinase, CALLA, CD10)], Genebank Accession Number: BC013875 [Matrix metallopeptidase 1 (interstitial collagenase)], Genebank Accession Number: BG284742 [P8 protein (candidate of metastasis 1)], Genebank Accession Number: AK124635 (Proprotein convertase subtilisin/kexin type 9), Genebank Accession Number: NM_{—}138711 (Peroxisome proliferative activated receptor, gamma), Genebank Accession Number: BX648997 (Peroxisome proliferative activated receptor, gamma, coactivator 1, beta), Genebank Accession Number: NM_{—}003621 [PTPRF interacting protein, binding protein 2 (liprin beta 2)],
Genebank Accession Number: XM_{—}350880 [Protein phosphatase 1H (PP2C domain containing)], Genebank Accession Number: BC033883 (Retinol binding protein 7, cellular), Genebank Accession Number: AL137502 (Ras-related GTP binding D), Genebank Accession Number: NM_{—}005063 [Stearoyl-CoA desaturase (delta-9-desaturase)], Genebank Accession Number: NM_{—}003627 (Solute carrier family 43, member 1), Genebank Accession Number: NM_{—}012449 (Six transmembrane epithelial antigen of the prostate 1), Genebank Accession Number: AL834346 [Syntaxin binding protein 6 (amisyn)], Genebank Accession Number: AL832142 [Transmembrane 7 superfamily member 1 (upregulated in kidney)], Genebank Accession Number: NM_{—}003820 [Tumor necrosis factor receptor superfamily, member 14 (herpesvirus entry mediator)], Genebank Accession Number: NM_{—}033035 (Thymic stromal lymphopoietin), Genebank Accession Number: AW665665 [Transcribed locus, strongly similar to XP_{—}509406.1 PREDICTED: similar to hypothetical protein FLJ14627 (Pan troglodytes)], Genebank Accession Number: NM_{—}003377 (Vascular endothelial growth factor B), EnsEMBL Accession Number: ENST00000313481.

23. The method according to claim 21, wherein the group of genes contains at least one gene selected from the group consisting of:
Genebank Accession Number: NM_{—}170589 (Cancer susceptibility candidate 5), Genebank Accession Number: BC053619 (Arrestin domain containing 3), Genebank Accession Number: NM_{—}000046 (Arylsulfatase B), Genebank Accession Number: AY367065 [Asp (abnormal spindle)-like, microcephaly associated (Drosophila)], Genebank Accession Number: NM_{—}052876 [BTB (POZ) domain containing 14B], Genebank Accession Number: NM_{—}031966 (Cyclin B1), Genebank Accession Number: NM_{—}001813 (Centromere protein E, 312 kDa), Genebank Accession Number: BC036307 (Calponin 1, basic, smooth muscle), Genebank Accession Number: M28016 (Human mitochondrial cytochrome b gene, partial cds.), Genebank Accession Number: BC065304 (DEP domain containing 1), Genebank Accession Number: AB209653 [Dehydrogenase/reductase (SDR family) member 2], Genebank Accession Number: NM_{—}001964 (Early growth response 1), Genebank Accession Number: AK122613 (ATPase type 13A5), Genebank Accession Number: CR600908 [full-length cDNA clone CS0DL005YJ22 of B cells (Ramos cell line) Cot 25-normalized of Homo sapiens(human).], Genebank Accession Number: BC043371 [Growth factor independent 1B (potential regulator of CDKN1A, translocated in CML)], Genebank Accession Number: NM_{—}016548 (Golgi phosphoprotein 2), Genebank Accession Number: NM_{—}005319 (Histone 1, H1c), Genebank Accession Number: NM_{—}003512 (Histone 1, H2ac), Genebank Accession Number: BC082232 (Histone 1, H2bg), Genebank Accession Number: BC101655 (Histone 1, H2bi), Genebank Accession Number: NM_{—}080593 (Histone 1, H2bk), Genebank Accession Number: BM752802 (Histone 1, H2bm), Genebank Accession Number: BX647290 (Histone 1, H2bo), Genebank Accession Number: BC069193 (Histone 2, H2be), Genebank Accession Number: AF032862 [Hyaluronan-mediated motility receptor (RHAMM)], Genebank Accession Number: BE620675 [Transcribed locus, moderately similar to NP_{—}536855.1 cytochrome b (Homo sapiens)], Genebank Accession Number: AY358369 [PRO333; Homo sapiens clone DNA41374 SIGLEC5 (UNQ294) mRNA, partial cds.], Genebank Accession Number: BE300829 [Transcribed locus, moderately similar to NP_{—}005021.2 polo-like kinase; polo (Drosophila)-like kinase; polo-like kinase (Drosophila) (Homo sapiens)], Genebank Accession Number: AY791349 (Kinesin family member 18A), Genebank Accession Number: AK025790 (Kinesin family member 20A), Genebank Accession Number: BC029844 (Hypothetical protein LOC256021), Genebank Accession Number: AF001540 [metastasis associated lung adenocarcinoma transcript 1 (non-coding RNA)], Genebank Accession Number: NM_{—}001620 [AHNAK nucleoprotein (desmoyokin)], Genebank Accession Number: NM_{—}00593 [Myeloid/lymphoid or mixed-lineage leukemia (trithorax homolog, Drosophila)], Genebank Accession Number: NM_{—}012333 (C-myc binding protein), Genebank Accession Number: AJ002535 (Obscurin, cytoskeletal calmodulin and titin-interacting RhoGEF), Genebank Accession Number: AB209179 [Polo-like kinase 1 (Drosophila)], Genebank Accession Number: NM_{—}006904 (Protein kinase, DNA-activated, catalytic polypeptide), Genebank Accession Number: BC050630 [RAB3 GTPase activating protein subunit 2 (non-catalytic)], Genebank Accession Number: AB095943 (SNF2 histone linker PHD RING helicase), Genebank Accession Number: AB016092 (Serine/arginine repetitive matrix 2), Genebank Accession Number: NM_{—}006472 (Thioredoxin interacting protein), Genebank Accession Number: DQ097177 (HECT, UBA and WWE domain containing 1), Genebank Accession Number: NM_{—}016267 [Vestigial like 1 (Drosophila)], TIGR Accession Number: THC2428713.
